(19) United States
(12) Patent Application Publication
(10) Pub. No.: US 2021/0161286 A1
(43) Pub. Date: Jun. 3, 2021

(54) Silver-Sterilizing Sponge Tooth Brushing Appliance, Silver-Sterilizing Toothbrush And Method Of Manufacturing The Same

(71) Applicant: Silver Future Co., Ltd., Kawaguchi-shi (JP)

(72) Inventor: Akira HIRAI, Kawaguchi-shi (JP)

(21) Appl. No.: 16/792,435

(22) Filed: Feb. 17, 2020

(30) Foreign Application Priority Data
Nov. 28, 2019 (KR) 10-2019-0156195

(51) Int. Cl. A46D 1/00; A46B 9/00; A46B 9/04; A46B 15/00; A46D 1/04; A61L 2/00; A61L 2/26

(52) U.S. Cl. CPC A46D 1/006; A46B 9/005; A46B 9/04; A46B 15/00; A61L 2202/17; A61L 2/0082; A61L 2/26; A46B 2200/1066; A46D 1/04

(57) ABSTRACT

A silver-sterilizing sponge tooth brushing appliance, a silver-sterilizing toothbrush, and a method of manufacturing the same are provided. More particularly, a silver-sterilizing sponge tooth brushing appliance containing one or two or more particles selected from talc particles, ceramic particles, and inorganic mineral particles, which have an sintered silver-containing surface layer, a silver-sterilizing toothbrush, and a method of manufacturing the same. The silver-sterilizing sponge tooth brushing appliance and the silver-sterilizing toothbrush have an excellent sterilizing effect.

### CROSS-REFERENCE TO RELATED APPLICATION

This application claims priority to and the benefit of Korean Patent Application No. 2019-0156195, filed Nov. 28, 2019, the disclosure of which is incorporated herein by reference in its entirety.

### BACKGROUND

### 1. Field of the Invention

The present invention relates to a silver-sterilizing sponge tooth brushing appliance, a silver-sterilizing toothbrush, and a method of manufacturing the same, and more particularly, to a silver-sterilizing sponge tooth brushing appliance containing one or two or more particles selected from talc particles, ceramic particles, and inorganic mineral particles, which have a sintered silver-containing surface layer, a silver-sterilizing toothbrush containing one or two or more particles selected from ceramic particles, and inorganic mineral particles, and a method of manufacturing the same. The silver-sterilizing sponge tooth brushing appliance and the silver-sterilizing toothbrush according to the present invention have an excellent sterilizing effect.

### 2. Discussion of Related Art

In dental medicine, various oral diseases including dental caries, gingivitis and periodontitis may occur by various causes. The causes may be classified into systemic causes and local causes, and the local causes are known as critical causes. The critical cause of the local causes is bacteria that reside in the oral cavity, that is, pathogenic microorganisms.

Meanwhile, tens of thousands of microorganisms are widely inhabited in various types of ecosystems in nature, and these microorganisms are also responsible for various diseases and contaminations. For example, in the oral cavity of a human, it has been known so far that about 300 types of microorganisms reside on the tooth surface, between teeth and gum at a dental root and on the surface of the tongue, and the presence of these microorganisms may be a normal phenomenon when appropriate oral hygienic activities are performed.

However, when appropriate oral hygienic activities are not performed, among the above-mentioned microorganisms, by pathogenic microorganisms, various oral diseases such as dental caries, halitosis, gingivitis or periodontal disease may be caused, and when the diseases become serious, teeth may be lost. As an example, dental caries may occur through breakdown of the enamel of the hard tissues of teeth due to lactic acids made by decomposing carbohydrates such as sugar or starch in food debris attached to the teeth due to the fermentation by pathogenic microorganisms residing in the oral cavity, and large-scale proliferation of anaerobic pathogens may be the cause of various oral diseases.

Pathological microorganisms residing in the oral cavity form bacterial populations called dental plaque only a few hours after attaching to the dental surface and are proliferated. At first, while the dental plaque is formed when the pathological microorganisms are intensively attached on the tooth surface at the upper part of the gum which is seen with eyes, as the formation of the plaque is gradually progressing, dental plaque is also formed on the tooth surface at the lower part of the gum. When the dental plaque is formed on the tooth surface, the pathological microorganisms produce acids using sugar entering the oral cavity, and the acids may break down minerals which are the main components of a tooth and thus break teeth, resulting in dental caries.

Meanwhile, bacteria residing in dental plaque at the lower part of the gum, particularly, anaerobic, Gram-negative microorganisms, secrete toxins and proteolytic enzymes to directly break down periodontal tissues or react with immune cells of the human body, thereby inducing the production of various immune materials, and these materials may lead to inflammation and breakdown of periodontal tissues.

There is the seriousness in that teeth and periodontal tissues can be treated once being destroyed, but they are not restored to their original tissue condition, and for this reason, fundamental blocking of the process of growing pathological microorganisms, which is the representative local cause of the destruction, in the oral cavity may be a method of preventing or rapidly treating an oral disease. Examples of the microorganisms causing such oral diseases include Streptococcus mutans, Porphyromonas gingivalis, etc. The Streptococcus mutans is the causative bacterium of dental caries, which causes various oral diseases. Particularly, glucan and lactic acid produced during the growth of Streptococcus mutans destruct the enamel of the teeth and generate plaque, thereby causing dental caries.

In recent years, there is a demand for development of toothbrushes that maintain oral hygiene for the elderly or infants and have a sterilizing effect.

### SUMMARY OF THE INVENTION

Conventional toothbrushes are intended to maintain oral hygiene, and are not able to give a sterilizing effect. Therefore, one object of the present invention is to provide a silver-sterilizing sponge tooth brushing appliance suitable for subjects such as the elderly or infants, and the like, a silver-sterilizing toothbrush, and a method of manufacturing the same.

However, the technical objects of the present invention are not limited thereto, and other objects of the present invention which are not stated herein will become more apparent to those of ordinary skill in the art by describing in detail exemplary embodiments thereof.

According to one aspect of the present invention, there is provided a silver-sterilizing sponge tooth brushing appliance including a silver-sterilizing sponge and a stick, wherein the stick is inserted into one side of the silver-sterilizing sponge, the silver-sterilizing sponge includes one or two or more particles selected from talc particles, ceramic particles, and inorganic mineral particles, wherein the talc particles, the ceramic particles, and the inorganic mineral particles have a sintered silver-containing surface layer formed thereon, and the one or two or more particles selected from the talc particles, the ceramic particles, and the inorganic mineral particles, which have the sintered silver-containing surface layer formed thereon, are dispersed inside and on a surface of the silver-sterilizing sponge.

According to another aspect of the present invention, there is provided a method of manufacturing a silver-sterilizing sponge tooth brushing appliance, comprises forming a silver-sintered film on surfaces of one or two or more particles selected from talc particles, ceramic particles, and inorganic mineral particles; preparing a silver-sterilizing sponge; preparing a tooth brushing appliance using the silver-sterilizing sponge, wherein the forming of the silver-sintered film on the surfaces of the one or two or more particles selected from the talc particles, the ceramic particles, and the inorganic mineral particles comprise a first step of preparing a silver salt solution by adding a silver salt compound powder to water and dissolving the silver salt compound powder therein; a second step of applying the silver salt solution onto the one or two or more particles selected from the talc particles, the ceramic particles, and the inorganic mineral particles; and a third step of sintering the silver salt solution, which has been applied onto the one or two or more particles selected from the talc particles, the ceramic particles, and the inorganic mineral particles, under a nitrogen atmosphere or under an oxygen atmosphere to form a sintered silver-containing surface layer on the one or two or more particles selected from the talc particles, the ceramic particles, and the inorganic mineral particles, and wherein the one or two or more particles selected from the talc particles, the ceramic particles, and the inorganic mineral particles, which have the sintered silver-containing surface layer formed thereon, are dispersed inside and on a surface of the silver-sterilizing sponge.

According to another aspect of the present invention, there is provided a silver-sterilizing toothbrush including a silver-sterilizing brush and a stick, wherein the silver-sterilizing brush includes one or two or more particles selected from talc particles, ceramic particles, and inorganic mineral particles, wherein the talc particles, the ceramic particles, and inorganic mineral particles have a sintered silver-containing surface layer formed thereon, and the one or two or more particles selected from the talc particles, the ceramic particles, and the inorganic mineral particles, which have the sintered silver-containing surface layer formed thereon, are dispersed inside and on a surface of the silver-sterilizing brush.

According to another aspect of the present invention, there is provided a method of manufacturing a silver-sterilizing toothbrush, comprises forming a silver-sintered film on surfaces of one or two or more particles selected from talc particles, ceramic particles, and inorganic mineral particles; preparing a silver-sterilizing brush; and preparing a toothbrush using the silver-sterilizing brush, wherein the forming of the silver-sintered film on the surfaces of the one or two or more particles selected from the talc particles, the ceramic particles, and the inorganic mineral particles comprises a first step of preparing a silver salt solution by adding a silver salt compound powder to water and dissolving the silver salt compound powder therein; a second step of applying the silver salt solution onto the one or two or more particles selected from the talc particles, the ceramic particles, and the inorganic mineral particles; and a third step of sintering the silver salt solution, which has been applied onto the one or two or more particles selected from the talc particles, the ceramic particles, and the inorganic mineral particles, under a nitrogen atmosphere or under an oxygen atmosphere to form a sintered silver-containing surface layer on the one or two or more particles selected from the talc particles, the ceramic particles, and the inorganic mineral particles, and wherein the one or two or more particles selected from the talc particles, the ceramic particles, and the inorganic mineral particles, which have the sintered silver-containing surface layer formed thereon, are dispersed in the inside and on a surface of the silver-sterilizing brush.

### BRIEF DESCRIPTION OF THE DRAWINGS

The above and other objects, features and advantages of the present invention will become more apparent to those of ordinary skill in the art by describing in detail exemplary embodiments thereof with reference to the attached drawings, in which:

FIG. 1 is a diagram showing a silver-sterilizing sponge tooth brushing appliance according to the present invention;

FIG. 2 is a diagram showing a method of manufacturing a silver-sterilizing sponge tooth brushing appliance according to the present invention;

FIG. 3 is a diagram showing a silver-sterilizing toothbrush according to the present invention; and

FIG. 4 is a diagram showing a method of manufacturing a silver-sterilizing toothbrush according to the present invention.

### DETAILED DESCRIPTION OF EXEMPLARY EMBODIMENTS

Hereinafter, exemplary embodiments of the present invention will be described in detail. However, the present invention is not limited to the embodiments disclosed below and can be implemented in various forms. The following embodiments are described in order to enable those of ordinary skill in the art to embody and practice the present invention.

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of exemplary embodiments. The term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

The terminology used herein is for the purpose of describing particular embodiments only and is not intended to be limiting of exemplary embodiments. The singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, components and/or groups thereof, but do not preclude the presence or addition of one or more other features, integers, steps, operations, elements, components and/or groups thereof.

With reference to the appended drawings, exemplary embodiments of the present invention will be described in detail below. To aid in understanding the present invention, like numbers refer to like elements throughout the description of the figures, and the description of the same elements will be not reiterated.

Silver-Sterilizing Sponge Tooth Brushing Appliance and Method of Manufacturing the Same

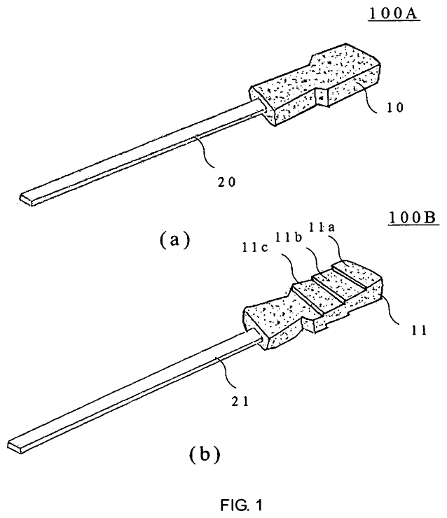

FIG. 1 is a diagram showing a silver-sterilizing sponge tooth brushing appliance according to the present invention.

FIG. 1A is a diagram showing one embodiment of a silver-sterilizing sponge tooth brushing appliance 100A according to the present invention. The silver-sterilizing sponge tooth brushing appliance 100A includes a silver-sterilizing sponge 10 and a stick 20. The stick 20 is inserted into one side of the silver-sterilizing sponge 10. The silver-sterilizing sponge 10 includes one or two or more particles selected from talc particles, ceramic particles, and inorganic mineral particles. A sintered silver-containing surface layer is formed on surfaces of the particles selected from talc particles, ceramic particles, and inorganic mineral particles.

FIG. 1B is a diagram showing another embodiment of a silver-sterilizing sponge tooth brushing appliance 100B according to the present invention. The silver-sterilizing sponge tooth brushing appliance 100B includes a silver-sterilizing sponge 11 and a stick 21. The stick 21 is inserted into one side of the silver-sterilizing sponge 11. In this case, because stepped portions 11a , 11b, and 11c are provided on the front and rear surfaces of the silver-sterilizing sponge 11, one can more thoroughly brush gaps between teeth. The silver-sterilizing sponge 11 includes one or two or more particles selected from talc particles, ceramic particles, and inorganic mineral particles . A sintered silver-containing surface layer is formed on surfaces of the particles selected from talc particles, ceramic particles, and inorganic mineral particles.

In particular, the one or two or more particles selected from the talc particles, the ceramic particles, and the inorganic mineral particles are dispersed on a surface of the silver-sterilizing sponge 10 or 11, as well as inside the silver-sterilizing sponge 10 or 11. Therefore, silver ions are eluted in water to exhibit a sterilizing effect. Also, when the inside of the mouth is brushed with the silver-sterilizing sponge 10 or 11, plaque may be removed by the one or two or more particles selected from the talc particles, the ceramic particles, and the inorganic mineral particles, which are dispersed on the surface of the silver-sterilizing sponge 10 or 11.

Hereinafter, a specific method of manufacturing a silver-sterilizing sponge tooth brushing appliance will be described.

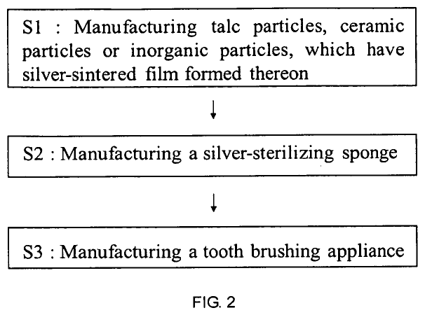

FIG. 2 is a diagram showing a method of manufacturing a silver-sterilizing sponge tooth brushing appliance according to the present invention. The method of the present invention includes forming a silver-sintered film on surfaces of one or two or more particles selected from talc particles, ceramic particles, and inorganic mineral particles (S1), preparing a silver-sterilizing sponge (S2), and preparing a tooth brushing appliance using the silver-sterilizing sponge (S3).

The forming of the silver-sintered film on the surfaces of the one or two or more particles selected from the talc particles, the ceramic particles, and the inorganic mineral particles (S1) according to the present invention includes a first step of preparing a silver salt solution by adding a silver salt compound powder to water and dissolving the silver salt compound powder in the water (S11); a second step of applying the silver salt solution on the one or two or more particles selected from the talc particles, the ceramic particles, and the inorganic mineral particles (S12); and a third step of sintering the silver salt solution, which has been applied onto the one or two or more particles selected from the talc particles, the ceramic particles, and the inorganic mineral particles, under a nitrogen atmosphere or under an oxygen atmosphere to form a sintered silver-containing surface layer on the one or two or more particles selected from the talc particles, the ceramic particles, and the inorganic mineral particles (S13).

In the above, talc particles, the ceramic particles, or the inorganic mineral particles, which are thermally resistant at a temperature of 500° C. or higher, are used as the sintered silver-coated particles.

The silver salt compound may be selected from silver carbonate, silver chlorate, silver chloride, silver chromate, silver vanadate, silver manganate, silver nitrate, silver nitrite, silver perchlorate, silver phosphate, silver acetate, etc., and a mixture thereof. Here, silver nitrate having the following Formula 1 may be preferably used.

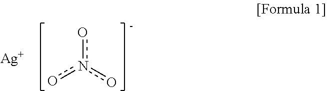

In this case, a heating process is required to apply a silver salt solution of silver nitrate to form a sintered silver-containing surface layer. Silver nitrate has a melting point of 212° C., but the silver nitrate has a decomposition temperature of 440° C. Therefore, 440° C. to 500° C. may be used as the sintering temperature. Accordingly, the sintered silver-containing surface layer may have excellent sterilizing properties.

A thickness of the silver-containing surface layer sintered under a nitrogen atmosphere or under an oxygen atmosphere is preferably in a range of 0.1 μm to 100 μm, and more preferably in a range of 10 μm to 80 μm. In nitrogen atmosphere, a content of nitrogen is in a range of 99% by volume to 100% by volume. Preferably, the content of nitrogen is in a range of 100% by volume. Also, the oxygen atmosphere refers to an atmosphere under which the silver-containing surface layer is sintered in the air.

A weight ratio of the silver-containing surface layer sintered under the nitrogen atmosphere or under the oxygen atmosphere to the one or two or more particles selected from the talc particles, the ceramic particles, and the inorganic mineral particles is preferably in a range of 0.1 to 50:100, and more preferably in a range of 1 to 40:100. Rapid sterilizing properties may be realized within this range.

The manufacturing of the silver-sterilizing sponge (S2) includes mixing base materials (S21); stirring the mixed base materials (S22); aging the stirred base materials (S23), curing the aged base materials (S24); cooling the cured base materials (S25); and cutting the base materials into pieces with an appropriate size (S26).

The mixing of the base materials (S21) includes mixing the one or two or more particles selected from the talc particles, the ceramic particles, and the inorganic mineral particles, which have a silver-sintered film formed thereon, with polyester polyol, polyethylene polyol, or a mixture thereof, an isocyanate, a pigment, a foaming agent, a curing agent, and the like. Here, the one or two or more particles selected from the talc particles, the ceramic particles, and the inorganic mineral particles, which have a silver-sintered film formed thereon, are preferably included in a content of 1% by weight to 50% by weight, and more preferably 5% by weight to20% by weight, based on the total weight of the mixed base materials. When the content is less than this range, a sterilizing effect or a plaque-removing effect cannot be expected. On the other hand, when the content is greater than this range, the prepared polyurethane sponge may become hard, and the one or two or more particles selected from the talc particles, the ceramic particles, and the inorganic mineral particles may aggregate.

The other base materials may be used at a known mixing ratio for a polyurethane sponge. The pigment is used to allow the prepared polyurethane sponge to have color.

The stirring of the mixed base materials (S22) included putting the base materials into a stirring machine, and uniformly mixing the base materials while stirring at 10 rpm to 100 rpm.

The aging of the stirred base materials (S23) includes processing the stirred base materials at 20° C. to 30° C. for 1 hour to 3 hours. When the stirred base materials are aged in this range, small microbubbles in the resulting mixture may grow into large bubbles, thereby regularly forming bubbles in a sponge.

The curing of the aged base materials (S24) includes putting the aged base materials into a curing machine and processing the base materials at 120° C. to 140° C. for 30 minutes to 1 hour. The curing agent may be evaporated in this process to give an elastic force to the polyurethane sponge.

The cooling of the cured base materials (S25) includes gradually cooling the cured base materials to room temperature.

The cutting of the base materials into pieces with an appropriate size (S26) includes cutting the polyurethane sponge into pieces with an appropriate size at which it is easy to put the silver-sterilizing sponge in the mouth and also to brush teeth with the silver-sterilizing sponge. To fix the stick inside the polyurethane sponge, narrow and long holes are also formed in sides of the polyurethane sponge.

In the manufacturing of the silver-sterilizing sponge (S2), the one or two or more particles selected from the talc particles, the ceramic particles, and the inorganic mineral particles, which have a sintered silver-containing surface layer formed thereon, are dispersed inside and on a surface of the silver-sterilizing sponge.

The manufacturing of the tooth brushing appliance using the silver-sterilizing sponge according to the present invention (S3) includes preparing a stick (S31); and fixing the stick in the silver-sterilizing sponge (S32).

The preparing of the stick (S31) includes preparing a thin and long stick in order to insert a stick into the obtained polyurethane sponge to brush teeth. Wood and plastics may be used as a material for the sticks.

The fixing of the stick in the silver-sterilizing sponge (S32) includes fixing a thin and long stick in a narrow and long hole formed in a side of the polyurethane sponge. To fix the stick in the side of the polyurethane sponge, an adhesive may be applied to the stick, and a cross section of the stick may be larger than a cross section of the hole formed in the side of the polyurethane sponge so that the stick can be physically fixed in the silver-sterilizing sponge.

In the above example, a polyurethane sponge that is excellent in a processing aspect is exemplified. However, a material including one or more selected from the group consisting of styrene butadiene rubber (SBR), a natural rubber (NR), latex, silicone, and a silicone-based elastomer may be used.

When the silver-sterilizing sponge of the present invention is used for the elderly or infants, oral hygiene may be maintained by dipping the silver-sterilizing sponge in water and brushing the mouth.

Also, the particles in which a silver-sintered film is formed on a surface of or inside the silver-sterilizing sponge are allowed to come into contact with water, and silver ions thus generated may be diffused in water, thereby exerting a sterilizing effect in the mouth. This is because the silver-sterilizing sponge has lots of holes by nature, thus the area of silver-sintered film of the particles is easily contacted with water. In addition, teeth are generally brushed approximately one to three times a day. In this case, after the silver-sterilizing sponge is used, the silver-sterilizing sponge is washed with water, and then stored. When the silver-sterilizing sponge is used the next time, water remaining in the silver-sterilizing sponge contains a large amount of silver ions because the water comes into contact with the particles having a silver-sintered film formed thereon for a long time. Therefore, the sterilizing effect is highly improved.

Also, because the particles having a silver-sintered film formed thereon are dispersed on a surface of the silver-sterilizing sponge, plaque is effectively removed when teeth are brushed with the silver-sterilizing sponge.

Silver-Sterilizing Toothbrush and Method of Manufacturing the Same

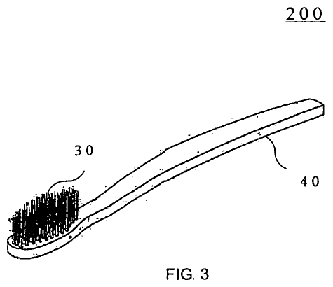

FIG. 3 is a diagram showing a silver-sterilizing toothbrush according to the present invention.

FIG. 3 is a diagram showing one embodiment of a silver-sterilizing toothbrush 200 according to the present invention.

The silver-sterilizing toothbrush 200 of the present invention includes a silver-sterilizing brush 30 and a stick 40. The silver-sterilizing brush 30 includes one or two or more particles selected from talc particles, ceramic particles, and inorganic mineral particles, which have a sintered silver-containing surface layer formed thereon.

Hereinafter, a method of manufacturing a silver-sterilizing toothbrush according to the present invention will be described.

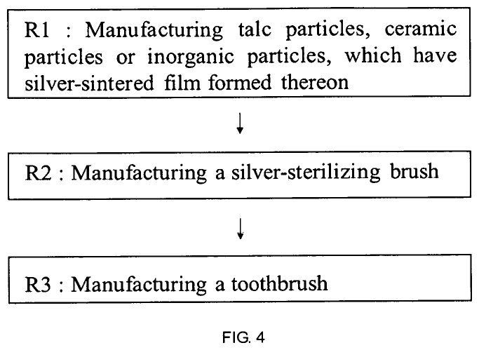

FIG. 4 is a diagram showing a method of manufacturing a silver-sterilizing toothbrush according to the present invention. The method of the present invention includes forming a silver-sintered film on one or two or more particles selected from talc particles, ceramic particles, and inorganic mineral particles (R1); preparing a silver-sterilizing brush (R2); and preparing a toothbrush using the silver-sterilizing brush (R3).

The forming of the silver-sintered film on the one or two or more particles selected from the talc particles, the ceramic particles, and the inorganic mineral particles (R1) is as described in the aforementioned method of manufacturing a silver-sterilizing sponge tooth brushing appliance.

The manufacturing of the silver-sterilizing brush (R2) includes mixing a polymeric resin, which is a compound selected from the group consisting of a polyurethane resin, a polybutylene terephthalate resin, and nylon, or a mixture thereof, one or two or more particles selected from talc particles, ceramic particles, and inorganic mineral particles, which have a silver-sintered film formed thereon, and a functional additive (R21); extruding the mixture through a nozzle of an extruding machine (R22); cooling and drying the extruded silver-sterilizing brush (R23); and cutting the silver-sterilizing brush to pieces with a certain size and polishing the silver-sterilizing brush (R24). Since, the silver-sintered film of the particles are partially and totally covered by the silver-sterilizing brush, the silver-sterilizing brush may be polished by surface removing process, such as sandblast process to the silver-sintered film being contacted with water.

In this case, the one or two or more particles selected from the talc particles, the ceramic particles, and the inorganic mineral particles, which have a silver-sintered film formed thereon, are preferably included in a content of 1% by weight to 50% by weight, and more preferably 5% by weight to 20% by weight, based on the total weight of the mixed base materials. When the content is less than this range, a sterilizing effect or a plaque-removing effect cannot be expected. On the other hand, when the content is greater than this range, the talc particles may aggregate in the brush.

The other base materials may be used at a known mixing ratio for a toothbrush.

The manufacturing of the toothbrush using the silver-sterilizing brush (R3) includes fixing a stick in the resulting silver-sterilizing brush. The fixing of the stick in the silver-sterilizing brush may be performed using methods known in the art.

When the silver-sterilizing toothbrush of the present invention is used, the silver-sterilizing toothbrush may have sterilizing properties while cleaning the teeth with the silver-sterilizing brush. That is, saliva or water in the mouth is allowed to come into contact with the silver-sterilizing brush to elute silver ions. As a result, the silver-sterilizing toothbrush may have a sterilizing effect. Also, when the silver-sterilizing toothbrush is washed after use, water may remain in the brush. In this case, this water contains a large amount of silver ions because the water comes into contact with the particles having a silver-sintered film formed thereon for a long time. Therefore, the sterilizing effect is improved.

Also, when the teeth are brushed with the particles having silver-sintered film formed thereon, which are dispersed on a surface of the silver-sterilizing brush, plaque is effectively removed.

Hereinafter, the present invention will be described in further detail with reference to an Experimental Example thereof. However, it should be understood that the present invention is not limited to the following Experimental Example.

### EXPERIMENTAL EXAMPLE 1

### Evaluation of Sterilizing Property According to the Present Invention

Next, a sterilizing property experiment for a ball having a sintered silver-containing coating layer of the present invention was performed. The experiment was carried out in the Japan Food Research Laboratories (JFRL). Porphyromonas gingivalis and Streptococcus nutans test bacteria were cultured in normal agar media (Eiken Chemical Co., Ltd.) at 35° C. (±1° C.) for 18 to 24 hours, and suspended in distilled water (a saline solution for Staphylococcus aureus) to have a cell density of 10⁷ to 10⁸ cells/mL, resulting in obtaining a test cell culture. Specimens are as follows. (Specimen) Ball having sintered silver-containing coating layer (Control) Distilled water contained in sterilized synthetic resin container (Test solution) A test solution was prepared by inoculating a sample prepared by adding 200 mL of mineral water to the specimen subjected to dry heat sterilization (170° C., 1 hour) with 2 mL of the test cell culture. Six hours and 24 hours after storage at 20° C. (±1° C.), the test solution was immediately diluted 10 fold using an SCDLP medium (Nihon Pharmaceutical Co., Ltd.), and then a viable cell count in the test solution was measured using a medium for measuring a bacterial cell count.

The test result is shown in Tables 1 and 2 below. The sterilizing property test showed that the specimen exhibited a sufficient sterilizing property effect on Porphyromonas gingivalis and Streptococcus mutans. Table 1 shows the result of measuring a viable cell count over time using Porphyromonas gingivalis test bacteria in the test solution, and Table 2 shows the result of measuring a viable cell count over time using Streptococcus mutans test bacteria in the test solution.

TABLE 1
Result of measuring viable cell count in test solution

| | | Viable cell count (/ml) | | | | | |
|---|---|---|---|---|---|---|---|
| | | | | After | After | After 2- | After |
| | Object for | After | After 5 | 10 | 15 | 20 | 25 |
| Test bacteria | measurement | initiation | minutes | minutes | minutes | minutes | minutes |
| Porphyromonas | Specimen* | — | 2.4 × 10⁵ | 30,000 | 10,000 | <10,000 | <10,000 |
| gingivalis | Control | 6.4 × 10⁵ | 3.0 × 10⁵ | 1.6 × 10⁵ | 1.1 × 10⁵ | 6.1 × 10⁵ | 1.8 × 10⁵ |

*(<10,000: indicating that the bacteria were not detected), Storage temperature: 20° C.

Referring to Table 1, it can be confirmed that the test solution of the present invention was tested to measure a viable cell count for Porphyromonas gingivalis, which was apparently different from the control from 5 to 10 minutes after the test. From 20 minutes after the test, it was confirmed that almost no viable cell count was detected.

TABLE 2
Result of measuring viable cell count in test solution

| | | Viable cell count (/ml) | | | | | |
|---|---|---|---|---|---|---|---|
| | Object for | After | After 5 | After 10 | After 15 | After 20 | After 25 |
| Test bacteria | measurement | initiation | minutes | minutes | minutes | minutes | minutes |
| Streptococcus | Specimen* | — | 4.7 × 10⁴ | 1.2 × 10⁴ | 2.4 × 10² | 30 | <10 |
| mutans | Control | 7.1 × 10⁵ | 6.3 × 10⁵ | 6.3 × 10⁵ | 6.0 × 10⁵ | 6.2 × 10⁵ | 6.2 × 10⁵ |

*(<10,000: indicating that the bacteria were not detected), Storage temperature: 20° C.

Referring to Table 2, it can be confirmed that the test solution of the present invention was tested to measure a viable cell count for Streptococcus nutans, which was apparently different from the control from 10 to 15 minutes after the test, and from 20 minutes after the test, it was confirmed that almost no viable cell count was detected.

To examine how talc particles having a sintered silver-containing coating layer affect the oral cavity, a silver ion detection test was performed. An aqueous solution of silver nitrate powder was used as a silver base material used herein. Also, talc particles (Food Additive Mineral No. 194) were used as the talc particles. First, 100 g of silver nitrate powder was added to 1 L of water to prepare a solution. Thereafter, talc particles were added thereto to prepare a silver solution mixture, sintered by heating at an average temperature of 450° C. under nitrogen of 99 vol % to 100 vol % to obtain talc particles coated with a silver-sintered film. Saliva in the mouth was subjected to a silver ion elution experiment. After 5 minutes, a content of the silver ions was 0.01 ppm, and, after 10 minutes, a content of the silver ions was 0.02 ppm. Therefore, a sterilizing effect of the silver ions against harmful bacteria (e.g., Gingivalis, Mutans, etc.) in the mouth was able to be confirmed by measuring a content of the silver ions.

The silver-sterilizing sponge tooth brushing appliance or the silver-sterilizing toothbrush according to the present invention includes one or two or more particles selected from talc particles, ceramic particles, and inorganic mineral particles, which have a surface layer containing sintered silver inside and on a surface thereof, and thus has an excellent sterilizing effect. Also, the silver-sterilizing sponge tooth brushing appliance or the silver-sterilizing toothbrush according to the present invention is useful in physically removing plaque while allowing one or two or more particles selected from talc particles, ceramic particles, and inorganic mineral particles, which have a surface layer containing sintered silver on a surface of the silver-sterilizing sponge or the silver-sterilizing toothbrush, to come into contact with teeth.

While the invention has been shown and described with reference to certain exemplary embodiments thereof, it will be understood by those skilled in the art that various changes in form and details may be made therein without departing from the spirit and scope of the invention as defined by the appended claims.

### BRIEF DESCRIPTION OF MAIN PARTS IN THE DRAWINGS

100A, 100B : silver-sterilizing sponge tooth brushing appliance

200: silver-sterilizing toothbrush

10, 11: silver-sterilizing sponges

20, 21: sticks for silver-sterilizing sponges

30: silver-sterilizing brush

40: stick for silver-sterilizing brushes

What is claimed is:

1. A silver-sterilizing sponge tooth brushing appliance comprising a silver-sterilizing sponge and a stick,
wherein the stick is inserted into one side of the silver-sterilizing sponge,
the silver-sterilizing sponge comprises one or two or more particles selected from talc particles, ceramic particles, and inorganic mineral particles, wherein the talc particles, the ceramic particles, and the inorganic mineral particles have a sintered silver-containing surface layer formed thereon, and
the one or two or more particles selected from the talc particles, the ceramic particles, and the inorganic mineral particles, which have the sintered silver-containing surface layer formed thereon, are dispersed inside and on a surface of the silver-sterilizing sponge.

2. A method of manufacturing a silver-sterilizing sponge tooth brushing appliance, comprising:
forming a silver-sintered film on surfaces of one or two or more particles selected from talc particles, ceramic particles, and inorganic mineral particles;
preparing a silver-sterilizing sponge;
preparing a tooth brushing appliance using the silver-sterilizing sponge,
wherein the forming of the silver-sintered film on the surfaces of the one or two or more particles selected from the talc particles, the ceramic particles, and the inorganic mineral particles comprises:
a first step of preparing a silver salt solution by adding a silver salt compound powder to water and dissolving the silver salt compound powder therein;
a second step of applying the silver salt solution onto the one or two or more particles selected from the talc particles, the ceramic particles, and the inorganic mineral particles; and
a third step of sintering the silver salt solution, which has been applied onto the one or two or more particles selected from the talc particles, the ceramic particles, and the inorganic mineral particles, under a nitrogen atmosphere or under an oxygen atmosphere to form a sintered silver-containing surface layer on the one or two or more particles selected from the talc particles, the ceramic particles, and the inorganic mineral particles, and
wherein the one or two or more particles selected from the talc particles, the ceramic particles, and the inorganic mineral particles, which have the sintered silver-containing surface layer formed thereon, are dispersed inside and on a surface of the silver-sterilizing sponge.

3. A silver-sterilizing toothbrush comprising a silver-sterilizing brush and a stick,
wherein the silver-sterilizing brush comprises one or two or more particles selected from talc particles, ceramic particles, and inorganic mineral particles, wherein the talc particles, the ceramic particles, and the inorganic mineral particles have a sintered silver-containing surface layer formed thereon, and
the one or two or more particles selected from the talc particles, the ceramic particles, and the inorganic mineral particles, which have the sintered silver-containing surface layer formed thereon, are dispersed inside and on a surface of the silver-sterilizing brush.

4. A method of manufacturing a silver-sterilizing toothbrush, comprising:
forming a silver-sintered film on surfaces of one or two or more particles selected from talc particles, ceramic particles, and inorganic mineral particles;
preparing a silver-sterilizing brush; and
preparing a toothbrush using the silver-sterilizing brush,
wherein the forming of the silver-sintered film on the surfaces of the one or two or more particles selected from the talc particles, the ceramic particles, and the inorganic mineral particles comprises:
a first step of preparing a silver salt solution by adding a silver salt compound powder to water and dissolving the silver salt compound powder therein;
a second step of applying the silver salt solution onto the one or two or more particles selected from the talc particles, the ceramic particles, and the inorganic mineral particles; and
a third step of sintering the silver salt solution, which has been applied onto the one or two or more particles selected from the talc particles, the ceramic particles, and the inorganic mineral particles, under a nitrogen atmosphere or under an oxygen atmosphere to form a sintered silver-containing surface layer on the one or two or more particles selected from the talc particles, the ceramic particles, and the inorganic mineral particles, and
wherein the one or two or more particles selected from the talc particles, the ceramic particles, and the inorganic mineral particles, which have the sintered silver-containing surface layer formed thereon, are dispersed in the inside and on a surface of the silver-sterilizing brush.
